(19) United States
(12) Patent Application Publication
(10) Pub. No.: US 2013/0006218 A1
(43) Pub. Date: Jan. 3, 2013

(54) Assisted Injection Device

(75) Inventors: Cyrille Vinchon, Chilly (FR); Vincent Guist'Hau, Cholet (FR)

(73) Assignee: ANTEIS SA, Plan-les-Ouates (CH)

(21) Appl. No.: 13/498,229

(22) PCT Filed: Sep. 27, 2010
(86) PCT No.: PCT/EP10/64225

§ 371 Date: Sep. 14, 2012

(30) Foreign Application Priority Data
Sep. 26, 2009 (EP) 09171427.9

(51) Int. Cl. A61M 5/20

(52) U.S. Cl. 604/506; 604/131

(57) ABSTRACT

Assisted injection device including a container for containing a product to be injected, the container including a movable bottom; a needle attached to the end of the container opposite the movable bottom; a piston acting on the movable bottom to expel through the needle a product located in the container and which is to be injected; a rotary motor for moving the piston by means of a drive mechanism, which converts the rotary motion of the rotary motor into a linear motion of the piston, the piston acting on the movable bottom by means of a compressible damping element.

The present invention relates to an assisted injection device. The present invention relates in particular to a device for assisting the injection of a cosmetic and/or therapeutic product for human and/or veterinary medical applications.

The increase of tissue volume, for example, can be desired both in the case of therapeutic applications and for a cosmetic purpose. It can be performed by the introduction of a viscoelastic solution on the basis of permanent or biodegradable products in the biological tissues. These products can exhibit very different viscoelasticity properties depending on the medical indication treated but also depending on the product formulation technology. There are thus highly functionalized products by reticulation or grafting of one or several polymers, having a single phase, reticulated or not, in gel form or in several phases, reticulated or not, sometimes integrating solid particles in the polymer matrix. Within the same technology, product ranges have been developed in order to answer the need of different indications, each of these products having different viscoelastic and flowing properties. These differences of properties are further exacerbated in that the associated means of administering them, be they needles, cannulas or syringe barrels, can be very different in terms of size, diameter or length, which reinforces the diversity available to the practitioner.

In the case of therapeutic applications, this type of viscoelastic solution is used for certain tissues that need to be enlarged to ensure their function: these are for example the vocal cords, the esophagus, sphincters or the urethra.

In the case of cosmetic applications, this type of viscoelastic solution is used for example to fill in wrinkles, to mask scars, to increase the volume of the lips, to remodel the shape of the face, the morphological reconstruction or the rejuvenation of the upper layers of the skin.

Very different injection techniques can be used depending on the indication treated. By way of example of frequently used injection techniques, the following can be mentioned: cross-hatching technique, or linear retrotracing technique, fan technique, anterotracing technique, multi-puncture technique (nappage), local deposit in significant quantity, papular or micropapular injection, serial puncture or multipoint technique. These techniques can be classified globally in two categories: continuous injection techniques and point-by-point injection techniques. These different techniques are not seldom combined during a same patient treatment session, depending on the treated indications.

According to the indication treated, the tissues can have very different resistance levels. In addition to the inter-patient physiological differences, the tissues of the upper dermis, of the middle or deeper dermis, of the hypodermis or the intramuscular zone or of the mucosae, as well as the deep periosteal or supra-periosteal tissues have very different density or laxity characteristics that will oppose variable resistance levels to the flow of the injected products. Each type of tissue considered individually cannot furthermore be considered a completely homogenous or amorphous medium. This is all the more so in the case of scar tissue, following prior trauma and a more or less fibrous reconstruction of these tissues.

The injection of a product in the body of a human being or of an animal is a delicate process requiring a certain know-how to prevent pain and other useless side effects for the patient and/or to dose the exact quantity of injected product.

This is particularly the case for the injection of products for a cosmetic purpose, especially for filler or rejuvenation products such as for example reticulated or non-reticulated gels on the basis of hyaluronic acid or of one of its salts. Indeed, it is particularly important during the injection of these products to control accurately the quantity of material injected at each place in order to achieve the desired cosmetic effect and avoid as much as possible the side effects associated with the needle perforation trauma or linked to the expansion or even dilacerations of the tissues during the injection. These side effects translate into pain during and after the injection and/or redness, irritations, edema, hematoma, inflammations, induration etc. after the injection of the product.

Depending on the degree of severity of the depression to be filled in the case of filler products or the degree of dryness of the skin associated to its type and thickness in the case of rejuvenation products, or depending on the severity of the pathology to be treated in the case of products with therapeutic indications, the trained medical practitioner knows how to judge the quantity of necessary product and the administration mode to be applied. However, the diversity in the products and administration means available, the variety of the techniques and of the depths of injection as well as the differences of tissue resistance make it difficult for the practitioners to dose the quantity of product.

In order to reduce the trauma associated with the perforation created by the needle as much as possible, the products are injected through fine needles, of a gauge comprised generally between 16 G and 36 G, more generally between 21 G and 32 G. This results in very high ejection forces, which makes it difficult to master the localization of the injection (site and depth) and the injected volume.

It must also be noted that the viscoelastic products used in the cosmetic and/or therapeutic indications in order to separate, replace, supplement or fill in soft tissues, or to increase their volume, are products that are sensitive to mechanical degradation and to the shearing that is characteristic for the extreme injection conditions and for the quick pressure variations induced by the resistance to the injection, whether it is linked to the tissues themselves or to the means of administration. All these products have viscosity curves according to the degree of shearing characterized by a quick drop of the viscosity properties.

Furthermore, it happens that the needle separates during the injection because of a continuous pressure that is too great or because of sudden changes of pressure in the syringe, due for example to a high resistance to the injection from the surrounding tissues. This phenomenon is all the more frequent during the injection of products with a high viscosity, such as for example certain products intended for cosmetic applications, and in particular when these products are injected through fine needles.

There are assisted injection devices in which the product to be injected is expelled out of the syringe by means of a fluid under pressure, of a spring or of an engine exerting a determined injection force on the mobile bottom of the syringe. Such devices do not however allow the rate or the quantity of the injected product to be satisfactorily regulated as the force necessary for expelling a certain quantity of product at a certain rate will depend on the resistance of the tissues in which the syringe's needle is inserted, on the choice of the administration means (needle, syringe) and on the viscoelastic properties of the injected product. The required injection force will thus be different for each patient, or even for each injection site on the same patient.

Because of the viscoelasticity properties and the high viscosity of the injected products, these assisted injection devices furthermore do not enable the flow to be stopped satisfactorily at the end of the deposit, which also interferes with the control of the quantity injected during the subsequent cycle. Simply stopping the thrust on the mobile bottom of the syringe does not make it possible to avoid the relaxation of the energy stored in the gel and a residual flow at the end of the needle. As to the exertion of a negative thrust (traction) on the mobile bottom of the syringe, it will nearly instantaneously create a depression in the gel and the aspiration of a volume of air at the end of the needle, which also interferes with the control of the quantity injected during the subsequent cycle. This difficulty of stopping accurately the flow of the injected product is all the more problematic in the cases of serial puncture injection where the injection of the product is frequently stopped.

Other assisted injection devices include a volumetric thrusting system, which affords them a better control of the quantity of injected product whatever the resistance of the tissues in which the injection takes place. Such a device also includes a mechanism enabling for example the rotation of an electric motor to be transformed into a linear progress of the syringe's bottom. This mechanism comprises for example a screw coupled to the rotation axis of the motor and turning in a rotationally fixed nut, thus causing the translation of the nut that is for example connected to the syringe bottom through a rigid piston. Thus, by accurately controlling the number of rotations performed by the motor and its rotational speed, it is possible to determine accurately the quantity of injected product and its rate, independently of the pressure at the exit of the syringe needle, of the choice of administration means (needle, syringe) or of the viscoelastic properties of the injected product.

One disadvantage of such devices is however that the direct mechanical connection between the motor and the bottom of the syringe sometime causes quick pressure changes in the syringe containing the product to be injected, which can cause pain to the patient and/or cause the needle to become unfastened, as explained further above.

One aim of the present invention is to propose a device for injecting cosmetic and/or therapeutic products, enabling the quantity and the rate of the injected product to be controlled accurately, whilst avoiding the pain and/or side effects suffered by the patients.

Another aim of the present invention is to propose a device for the assisted injection of cosmetic and/or therapeutic products enabling the risk of pain for the patient and/or of the needle becoming detached during the injection to be diminished as compared with the assisted injection devices of the prior art.

These aims are achieved by a device having the characteristics of the independent claim.

These aims are achieved in particular by an assisted injection device including a container for containing a product to be injected, the container including a movable bottom; a needle attached to the end of the container opposite the movable bottom; a piston acting on the movable bottom to expel through the needle a product located in the container and which is to be injected; a rotary motor for moving the piston by means of a drive mechanism, which converts the rotary motion of the rotary motor into a linear motion of the piston, the piston acting on the movable bottom by means of a compressible damping element.

The present invention will be better understood by reading the following description illustrated by the figures, where:

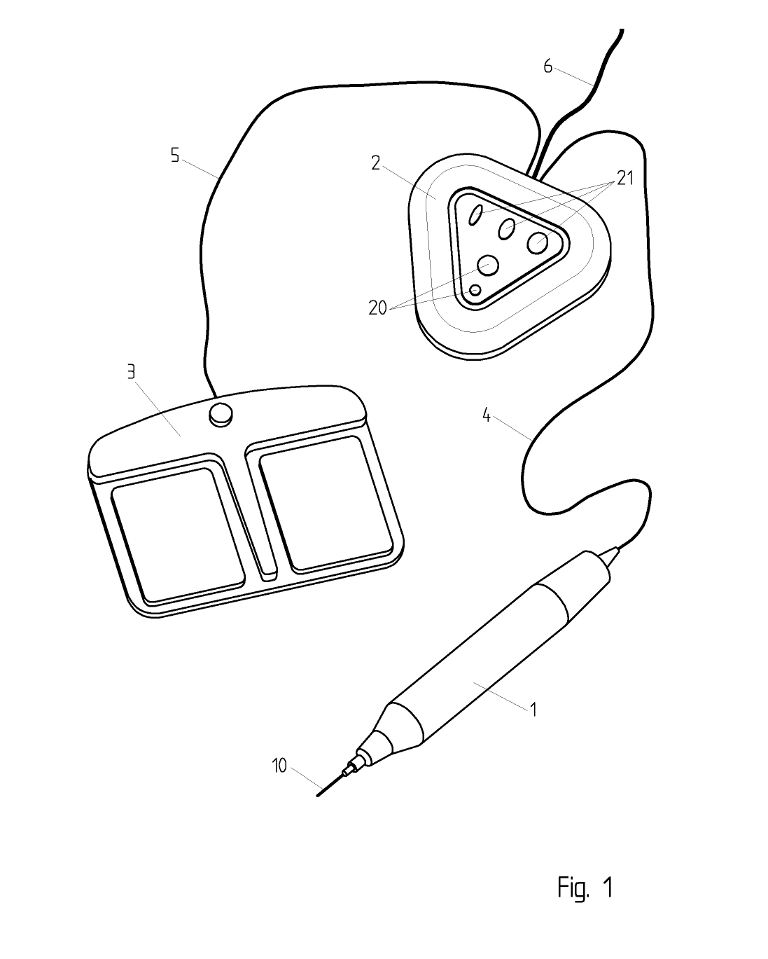

FIG. 1 represents an assisted injection device according to a preferred embodiment of the invention;

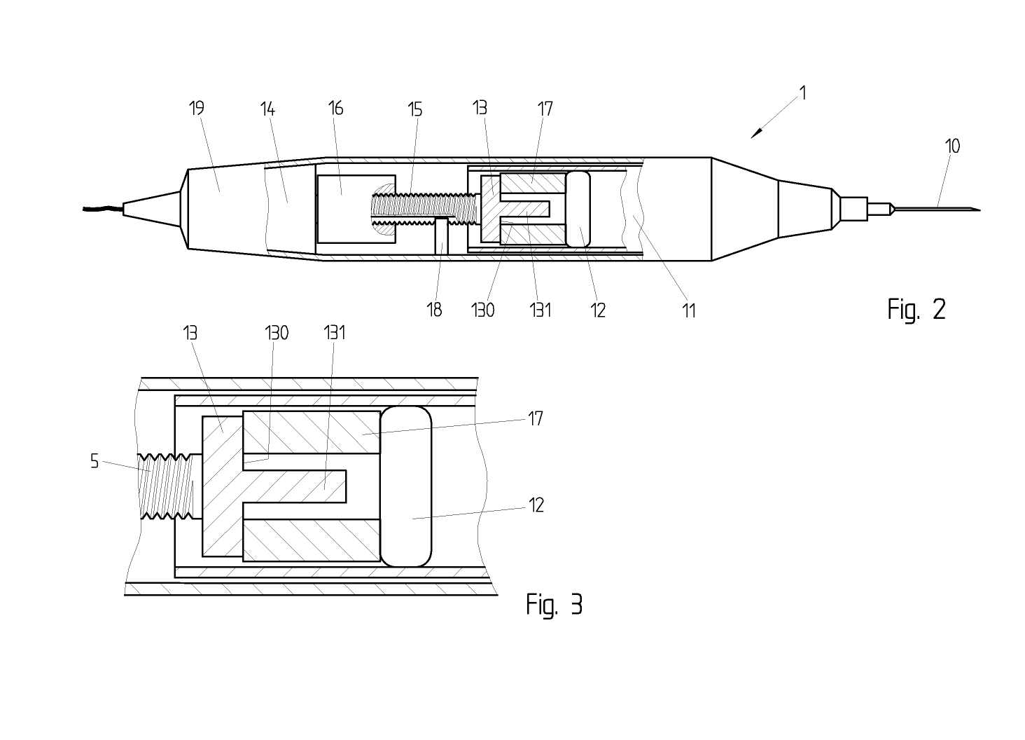

FIG. 2 is a partial cross-section view of the handpiece of the assisted injection device of FIG. 1;

FIG. 3 is a detail of FIG. 2;

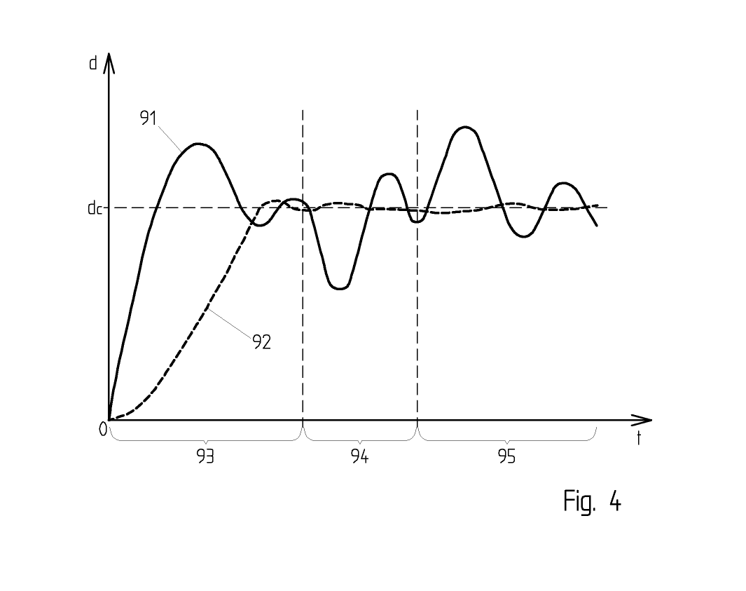

FIG. 4 illustrates the effects of the inventive device on the regulation of the rate during the injection.

With reference to FIG. 1, an assisted injection device typically comprises a handpiece 1, a control unit 2 and a foot pedal 3.

The handpiece 1, illustrated in more detail in FIG. 2, includes a container 11 designed to contain a product to be injected, onto which a needle 10 is fastened that enables the product contained in the container 11 to be injected. Preferably, the container 11 is elongated, for example cylindrical, and the needle 10 is fastened onto one of its two extremities, the other extremity of the container being closed by a mobile bottom 12. According to the invention, a piston 13 acting on the mobile bottom 12 of the container is driven by a rotary motor 14 by means of a drive mechanism to convert the rotary motion of the motor 14 into a translation motion of the piston 13, thus enabling the volume and the rate of the injected product to be controlled accurately.

According to a preferred embodiment of the invention represented by way of illustrative but non-limiting example in FIG. 2, the drive mechanism comprises a nut 16 rotating with the motor 14, driven for example directly by the axis of the motor 14, and a screw 15 at least partly screwed into the nut 16 and rotationally fixed relative to the motor 14, for example by means of a linear guiding pin 18. The linear guiding pin 18 is for example fastened to the inside wall of the housing 19 of the handpiece 1 and at least partly inserted for example in a groove provided along the screw 15, thus preventing the screw from turning relative to the housing 19 whilst allowing its translation motion relative to the latter. Thus, when the motor 14 rotates, it drives the nut 16, which turns in place, and the screw 15 is displaced in translation along its axis inside the handpiece 1. The linear guiding pin 18 is, for example, formed by molding during the manufacture of the housing 19.

The screw 15 is thus guided in its translation movement and is rotationally fixed inside the handpiece 1 whilst the nut 16 is blocked from translating and guided rotationally by the motor 14. The piston 13 acting on the mobile bottom 12 of the container 11 is then connected to the screw 15, which drives it in its translation motion. The piston 13 is preferably fixed, for example welded, to the extremity of the screw 15 so that the piston 13 is driven by the screw 15 in both directions along the axis of the screw 15. According to one variant embodiment, the piston 13 and the screw 15 are formed of a single part, for example by turning.

According to another embodiment, not represented, the screw is driven in rotation by the axis of the motor and blocked from translating, whilst the nut is rotationally fixed and free to move in translation in the handpiece along the screw's axis. The rotation of the motor, and thus of the screw, then causes the nut to move linearly inside the handpiece and the piston acting on the mobile bottom of the container is connected to the nut that drives it in its translation movement. The piston is then preferably fixed, for example welded, to the nut, or the piston and the nut are formed of a single part.

By knowing the thread of the screw 15, it is possible to determine exactly the amplitude and the speed of displacement of the piston 13 depending on the movements of the motor 14. In particular, the direction of displacement of the piston 13 depends on the rotational direction of the motor 14, the speed of displacement of the piston 13 depends on the rotational speed of the motor 14 and the amplitude of displacement of the piston 13 depends on the number of turns performed by the motor 14.

Thus, by accurately controlling the number of turns performed by the motor 14 and its rotational direction and speed, the quantity of injected product and the rate of injection can be determined and regulated accurately, regardless of the pressure at the exit of the needle 10, of the definition of the administration means and in particular of the size of the needle 10, or of the viscoelastic properties of the injected product.

The thread of the screw 15 is preferably small to enable the displacements of the piston 13 to be accurately regulated and to minimize the amplitude of the torque variations of the motor 14, and thus, for example, its electric current power supply, necessary to compensate the effects of the variations of the encountered resistance to the injection.

Other forms of the drive mechanism are however possible within the frame of the invention to transform the rotation of the motor 14 into a linear displacement of the piston 13. According to one embodiment, the drive mechanism comprises for example a toothed wheel driven by the motor and acting on a notched rod serving as piston. According to another embodiment, the drive mechanism comprises for example two parallel screws, a first screw being connected to the motor and driving rotationally the second screw that is displaced along a linear axis relative to the first screw.

The handpiece 1 preferably comprises a housing 19 of a rigid material and having an ergonomic shape, enabling the user to hold it well during the injection. The main elements of the handpiece 1 are preferably accommodated in the housing 19, with the exception of at least part of the needle 10.

The motor and the drive mechanism are preferably lodged in the handpiece 1. Other embodiments are however possible within the frame of the invention. According to one variant, the motor is lodged in the control unit in order to minimize the volume of the handpiece, and the drive mechanism acts on the piston, for example by means of a semi-rigid steel cable through the flexible connector connecting the control unit to the handpiece.

With reference to FIG. 1, the control unit 2 of the injection device includes for example a microprocessor, some memory and a software stored in the memory and which can be executed by the microprocessor, allowing it to control accurately the quantity and the rate of the product to be injected expelled from the handpiece 1 through the needle 10, by accurately controlling the movements of the motor, in particular its rotation direction, its rotation speed and the number of turns performed at each activation. The control unit 2 makes it possible for example to regulate the intensity and/or the voltage of the electric current supplied to the rotary electric motor in order to guarantee the motor's controlled movements that are regular and accurate, regardless of the encountered resistance to the injection.

The control unit 2 preferably comprises control buttons 20 allowing the user to select a rate and/or a quantity of product to be injected and/or to choose between a continuous or a drop-by-drop injection. The unit preferably also includes light indicators 21 and/or a digital display and/or a sound signaling device enabling the user to control visually and/or aurally the settings selected and/or the current dosage. During the injection, the software implemented in the control unit 2 ensures the motor functions regularly and accurately depending on the rate and/or quantity parameters entered previously by the user.

The activation of the motor is preferably controlled by means of a foot pedal 3, thus enabling the user to have both hands free to guide the insertion of the needle 10 under the patient's skin and possibly the movements of the needle 10 during the injection.

The handpiece 1, the control unit 2 and the food pedal 3 are preferably connected to one another by means of connectors that are preferably flexible, for example communication and/or supply cables 4, 5 enabling these elements to communicate, especially for command signals and/or control signals to be communicated and/or electric energy to be transmitted. The assisted injection device of the invention is preferably powered by an external electric energy source, not represented, to which it is connected for example by means of an electric power cable 6.

According to the invention, the piston acts on the mobile bottom of the container by means of a compressible damping element situated between the piston and the mobile bottom. A preferred embodiment of the damping element is illustrated in FIGS. 2 and 3 by way of illustrative but non-limiting example. The damping element 17 preferably has deformation abilities capable of absorbing the brutal pressure variations in the product contained in the container 11 and induced by the variations of resistance to the injection, whether they are linked to the tissues themselves or to the administration means. It also makes it possible if necessary to attenuate the accelerations of the motor, for example at the beginning of the injection or during sharp corrections of the thrust due to large variations of the encountered resistance to the injection.

According to the invention, in case of a quick increase of the pressure inside the container 11 during the injection, the damping element 17 situated between the mobile bottom 12 of the container 11 and the piston 13 is momentarily compressed, which momentarily slows the displacement of the mobile bottom 12 and thus avoids an overpressure in the container 11. The energy stored in the damping element 17 is then released to the mobile bottom 12 when the pressure in the container 11 has again decreased, for example when the resistance to the injection has diminished and/or the motor has stopped. The damping element 17 thus also makes it possible to reduce the degree of shearing of the product contained in the container 11 and thus to protect it against one form of degradation.

With reference to FIG. 3, the damping element 17 is preferably a flexible cylinder, for example of elastomer. The elastomer of the damping element 17 has for example a hardness comprised between 200 and 100 shore A, preferably between 50 and 80 shore A.

The extremity of the piston 13 preferably comprises a ledge 130 enabling the piston 13 to rest on the damping element 17 and optionally a central rod 131 capable of sliding freely inside the damping element 17. The ledge 130 and the central rod 131 are preferably formed of a single piece, for example by turning and/or molding of a cylindrical part of rigid material, for example of Teflon, of a metal etc.

According to one embodiment of the invention, the piston 13 also has a certain flexibility and thus also contributes to damping variations of pressure inside the container 11. The piston 13 is however preferably more rigid than the damping element 17.

According to one embodiment, the damping element 17 is an independent part inserted into the container and resting against the mobile bottom 12 of the container 11. According to another embodiment, the damping element 17 is fastened to the bottom of the container 11, being for example part of the same element as the mobile bottom 12. The damping element 17 and the mobile bottom 12 of the container 11 are then for example molded of a single piece in a flexible material, for example elastomer.

During the injection of the product contained in the container 11, the ledge 130 of the piston 13 rests on the side of the damping element 17 opposite to the mobile bottom 12. The deformation of the damping element 17 under this ledge makes it possible to attenuate the brutal pressure variations as regards the product to be injected and protects it. If the piston 13 comprises a central rod 131, the latter further makes it possible to limit the deformation of the damping element 17 by pressing directly on the mobile bottom 12 after a determined compression of the damping element 17. This has notably the effect of limiting the possible inaccuracies in the regulation of the rate or of the quantity of the product injected and which would be due to an excessive deformation of the damping element 17, and/or of limiting the involuntary flow of the product after the motor has stopped and which would be due to an excessive decompensating of the damping element 17.

Depending on the type of injection, the piston 13 is for example drawn backwards at the end of a series of injections, which allows the damping element 17 to be decompensated. This decompensating is accompanied by a return of the damping element 17 to its resting shape, by a quick release of the residual pressure in the product and by a stop of any residual flowing at the tip of the needle. According to a preferred embodiment of the invention, the piston 13 does not create a phenomenon of withdrawal of the mobile bottom 12 nor of aspiration since it does not drive the mobile bottom 12 when it returns, whilst the central rod 131, if present, slides freely in the damping element 17. The damping element 17 retrieves its shape at rest and moves back until the atmospheric pressure is re-equilibrated on each side of the product (on the needle side and on the mobile bottom 12 side), without air aspiration at the tip of the needle.

According to the invention, the damping element 17 enables, thanks to its compressibility, an initial progressive increase of the rate of injected product until the target rate has been reached, according to an increasing acceleration, thus minimizing the trauma of the tissues by getting them progressively used to the increase in volume.

The compressibility of the damping element 17 also enables the container 11 to be protected from brutal pressure changes, thus minimizing the risks of the needle becoming detached.

FIG. 4 illustrates by way of illustrative and non-limiting example the regulation of the rate of the injected product at the beginning of the injection and the regularity of the rate during the injection, achieved with the aid of the assisted injection device of the invention. The diagram of FIG. 4 thus shows the variation of the rate d of the injected product as a function of the time t between a rate zero at the beginning of the injection and a target rate d_{c}, using a manual injection or an injection assisted with the aid of the inventive device.

The curve 91 illustrates the typical variations in the rate of the product injected during a manual injection, where the practitioner attempts to regulate manually the displacement speed of the piston of a usual syringe in order to achieve a rate as regular and as close to the target rate d_{c} as possible. At the beginning of the injection, whilst the extremity of the injection needle is for example in a non-fibrous tissue 93 of the patient, the practitioner presses first quite strongly on the piston in order to quickly reach what he/she believes to be the target rate d_{c}, then attempts to stabilize the rate around this target rate d_{c}, generally after one or two oscillations. If the practitioner for example moves the extremity of the needle during the injection, which then penetrates for example into a zone of scar tissue 94 which is generally harder, the rate of injected product will tend to diminish and the practitioner will have to press harder on the syringe's piston to bring the rate close to the value of the target rate d_{c}. If the extremity of the needle penetrates for example again a zone of non-fibrous tissue 95, the rate will increase before the practitioner notices the change of resistance to the injection and can again attempt to correct the rate.

The curve 92 illustrates the typical variations in the rate of a product injected by means of an assisted injection device with volumetric control according to the invention, comprising a damping element. The curve 92 shows that at the beginning of the injection, i.e. when the motor of the handpiece starts and the piston initiates its linear advance, the rate of the product injected increases progressively by reason of the compression of the damping element, thus preventing any pain in the patient that would be due to a sudden change in rate and also limiting the risk of the needle becoming detached. Once the damping element has been compressed, the mobile bottom advances with the piston or even slightly faster because of the decompression of the damping element, so that the rate increases practically regularly until it reaches the target rate. The stabilizing of the injection rate around the target rate d_{c} is then relatively quick thanks to the volumetric control of the rate, which depends directly on the well-known and controlled movements of the motor, and the oscillations have a small amplitude and are at least partially due to the return of the damping element into its resting position or into another stable compression position. The curve 92 shows that once the target rate d_{c} has been reached, the rate remains essentially stable whatever the tissue into which the product is injected and its resistance to the injection.

The damping element is described according to the preferred embodiment of the invention in the shape of a cylinder of compressible matter. Other embodiments are however possible in the frame of the invention. According to an alternative embodiment, the damping element is for example a spring, for example a metallic spring, placed between the piston and the container's mobile bottom.

Confidential clinical tests, summarized in the example hereafter given by way of illustrative and non-limiting example, have demonstrated that the use of the assisted injection device according to the invention, comprising a damping element, for example of elastomer, between the piston and the mobile bottom, reduces the pain and the other side effects for the patient by comparison with an injection process using a manual thrust device.

### EXAMPLE

A clinical assessment of the assisted injection device of the invention, in particular in the frame of the injection of filler and rejuvenation products, was performed. The study was conducted by 8 practitioners in 7 countries.

One aim of the study was to supply clinical proof of the security and efficiency of the inventive device in the treatment of different facial wrinkles and in rejuvenation treatments. Another aim was to evaluate the strengths, weaknesses and ease of use of the inventive device.

In total, 193 patients were involved in this study. 130 patients received injections of products Mesolis or Mesolis+ in the frame of a rejuvenation treatment. 63 patients received injections of filler products Esthélis Soft, Esthélis Basic and/or Fortélis. In total, 267 indications were treated with the inventive device: 175 for rejuvenation and 92 with filler products.

The patients were sought by the practitioner to participate in this assessment when they consulted either to receive an injection or for a rejuvenation or for an injection of filler product, and sometimes for a combination of both treatments. For each patient having received an injection, the patient himself/herself and the practitioner had to fill in assessment forms and questionnaires. The practitioners performed these clinical tests under confidentiality agreement.

Result for the Filler Products

The principal indications were nasolabial folds and bitterness folds, which represented respectively 47% and 11% of all the indications.

Fortélis was the first filler product used, with 47% of the injections, just before Esthélis Basic with 45% of indications treated. Fortélis was used principally in continuous injection mode, or using the multi-puncture technique (nappage), in 62% of the cases, with a needle of gauge 27 G. The point-by-point or drop-by-drop injection mode was used in 38% of the treatments with Fortélis. Esthélis Basic and Esthélis Soft were used principally in continuous injection mode with a needle of gauge 30 G.

The continuous injection mode was used in more than 81% of the indications and the point-by-point injection mode in 19%.

Among the secondary effects reported, the main ones were redness and pain.

After the injection, the practitioners declared the result 100% satisfactory.

Certain patients had their nasolabial folds treated with both products, Fortélis and Esthélis Basic. Fortélis in continuous injection mode, in the deep dermis, and Esthélis Basic injected more superficially in continuous injection mode along the folds.

Among these patients treated with filler products, 63% had already received manual injections.

Most of them stated that the injection was not painful with the inventive device, but none of them said that they had felt more pain as compared with a manual injection.

The inventive device was used principally for the injection of Fortélis. This helps the injector to push the product into the dermis.

The main indication was the treatment of nasolabial folds. In this case, the inventive device was used in drop-by-drop injection mode at high speed and with a needle of gauge 27 G.

The use of the inventive device with filler products enables:
an injection with a better control of the rate and volume;
an injection that is less painful for the patient: quicker and more comfortable, with fewer side effects;
an easier injection of Fortélis, especially for deep folds;
a better result thanks to a better integration of the filler product into the dermis.

Result for Rejuvenation

The three main indications treated were firstly hydration of the face, at 65%, secondly crow's feet, at 12%, and thirdly the neck, at 7%. In these indications, the point-by-point injection mode was mainly used. For rejuvenation, Mesolis and/or Mesolis+ were used, principally in point-by-point injection mode and at low speed. The point-by-point injection mode was used for 88% of the indications. A needle of gauge 30 G was used in 52% of the cases. 46% of the injections were performed with a needle of gauge 32 G. The needles of gauge 32 G were used principally in point-by-point injection mode at low speed, for 62.5% of the cases. The needles of gauge 32 G are easy to use with the inventive device and the injection is less painful.

Use of an anesthetic before the treatment could be avoided and fewer side effects were observed.

For the principal indication relating to rejuvenation treatments, hydration of the face, an average of 1.2 ml of product was used for the entire face. The point-by-point injection mode at low speed and with needles of gauge 32 G were the most frequently used parameters. No anesthetic product was used in 74% of the cases.

Among the reported side effects, the main ones were slight redness and pain.

To conclude, use of the inventive device for revitalization treatments enables:
an injection with a better control of the speed and volume of the papules;
a use in both modes, continuous injection and point-by-point injection, in order to allow micro-papular and multi-puncture techniques;
a quicker return to social life with papules that disappear more quickly thanks to the homogenous volume of the drops of delivered product;
a less painful injection for the patient.

Conclusion

These results demonstrate that the inventive device is useful in the frame of injections of filler and rejuvenation products. The treatment by means of the inventive device enables a better control of the depth, of the rate and of the volume, and use of finer needles for the injection of rejuvenation products. Consequently, it is less painful for most of the patients; the product is positioned better and is more homogenous and better integrated in the dermis. It yields satisfactory results and very few negative consequences as compared with a manual injection.

In the case of revitalization, the anesthetic procedures could be avoided and the only side effects reported were slight redness and a light pain.

With filler products, the injection is easy and painless. The product is distributed in a very homogenous and natural manner in the dermis, more so than with a manual injection.

The assisted injection device of the invention is compatible with all injection techniques and all types of products, in particular revitalization and filler products.

The inventive device is easy to use since it is held like a pencil, between two fingers.

Patients have reported that they were satisfied with the treatment, that they had felt less pain with the inventive device as compared with a manual injection. Some of them reported that they enjoyed the noise made by the device and which seemed to provide a relaxing effect.

The practitioners appreciated the use of the device, as it is more accurate and reduces muscular fatigue. The volume of the drops is regular and very small papules can be produced. It allows the patients to return to their social life better and more quickly, since fewer side effects have been noticed.

The inventive device has been used with all types of products, rejuvenation and filler, with a high level of satisfaction, close to 100%.

Preferably, the container 11 and/or the needle 10 of the assisted injection device of the invention are replaceable and/or disposable.

The housing 19 of the handpiece 1 is formed for example of two parts assembled by screwing, which makes it thus possible to open the handpiece 1 and withdraw the container 11 after injection of the product to be injected contained therein in order to replace it with a filled container, of the same or of a different product to be injected. The empty or partially empty container is for example eliminated or filled again for a new use. The needle 10 is also replaceable in order to enable it to be eliminated or sterilized after the injection, and/or to enable it to be replaced by a clean and disinfected needle of the same or a different diameter, for a new injection.

When the container 11 is removed, the damping element 17 preferably remains with the container and is consequently also replaced. According to a variant embodiment, the damping element is kept when the container 11 is changed and it is then associated with the new container.

The assisted injection device of the invention is adapted for injecting any cosmetic and/or therapeutic product in the frame of human and/or veterinary medicine.

It is in particular adapted to the injection of a cosmetic and/or therapeutic product in soft intra-dermal, subcutaneous or periosteal tissues of a patient in order to separate, replace, supplement or fill in these tissues, or to increase their volume, which requires a particular accuracy in terms of the quantity of product injected and of its distribution in order to achieve satisfactory results. These applications generally require the injection of viscoelastic solutions whose dosage is greatly facilitated by the use of the assisted injection device of the invention. Furthermore, the injection of viscoelastic solutions often causes pain during sudden changes of the injection rate which are avoided thanks to the assisted injection device according to the invention.

The assisted injection device of the invention is adapted to its use in any method for filling and/or rejuvenating soft intra-dermal, subcutaneous or periosteal tissues of a patient, including the injection into said tissues of the patient of a filler and/or revitalization tissue. The inventive device notably enables the practitioner, by means of the control buttons on the control unit, to choose between a continuous injection and a drop-by-drop injection.

The assisted injection device of the invention is thus adapted to its use in any method for separating, replacing, filling in or supplementing soft intradermal, subcutaneous or periosteal tissues of a patient, including the steps of inserting the extremity of the needle 10 of the inventive device into said tissues of the patient and activating the rotary motor 14 of the device to displace the piston 13 by means of a drive mechanism 15, 16 transforming the rotary motion of said rotary motor 14 into a linear motion of said piston 13 to expel through said needle 10 a cosmetic and/or therapeutic product located in the container 11, wherein the variations in the rate of said cosmetic and/or therapeutic product through said needle 10 are absorbed by the damping element 17.

The assisted injection device of the invention is however also adapted for the injection of therapeutic and/or cosmetic products, including for low-viscosity products, for example in cartilage, articulations, veins, the intra-ocular cavity or the extra-ocular surgical cavity, for obturating for example veins, or for treating these elements thanks to a controlled salting out. The assisted injection device according to the invention is thus for example adapted for treatments in the fields of rheumatology, ophthalmology, phlebology, for the injection of toxins for example in the treatment of hyperhidrosis and/or for the multiple therapeutic treatment resulting from the injection of a sustained release drug delivery device, of an active agent or of a medicament.

More generally, the assisted injection device of the invention is adapted to its use in any therapeutic method including the injection of toxins, of an active agent or of a medicament into a cartilage, an articulation, a vein, an intra-ocular cavity or an extra-ocular surgical cavity of a patient. The injection can furthermore be a continuous injection or a point-by-point injection.

The assisted injection device of the invention is adapted to its use in any treatment method in the field for example of fields of rheumatology, ophthalmology, phlebology, for the treatment of hyperhidrosis or for the obturation of the vein of a patient, including the injection of toxins, of an active agent or of a medicament to the patient, including the steps of inserting the needle 10 of the inventive device into a cartilage, an articulation, a vein, an intra-ocular cavity or an extra-ocular surgical cavity of the patient, and activating the rotary motor 14 of said device for moving said piston 13 by means of a drive mechanism 15, 16, which converts the rotary motion of the rotary motor 14 into a linear motion of said piston 13 to expel through said needle 10 toxins, an active agent or of a medicament located in the container 11, wherein the variations in the rate of said toxins, active agent or medicament through said needle 10 are absorbed by said damping element 17.

1. Assisted injection device including:
a container for containing a product to be injected, said container including a movable bottom;
a needle attached to the end of said container opposite the movable bottom;
a piston acting on said movable bottom to expel through said needle a product located in said container and which is to be injected;
a rotary motor for moving said piston by means of a drive mechanism, which converts the rotary motion of said rotary motor (14) into a linear motion of said piston;
wherein said piston acts on the movable bottom by means of a compressible damping element.

2. The device of claim 1, wherein said piston comprises a ledge for resting onto said damping element.

3. The device of claim 2, wherein said piston further comprises a rod extending freely inside said damping element when said ledge rests onto said damping element to limit the deformation of said damping element.

4. The device of claim 1, wherein said damping element is cylindrical and placed in said container against said mobile bottom.

5. The device of claim 1, wherein said damping element is a cylinder of elastomer.

6. The device of claim 1, wherein said damping element has a hardness comprised between 20 and 100 shore A.

7. The device of claim 6, said damping element has a hardness comprised between 50 and 80 shore A.

8. The device of claim 1, wherein said damping element is a spring.

9. The device of claim 8, wherein said damping element is a metallic spring.

10. The device of claim 1, wherein said damping element is fastened to said mobile bottom.

11. The device of claim 1, wherein said container is replaceable by another container.

12. The device of claim 1, wherein said needle is replaceable by another needle.

13. A method for separating, replacing, filling or supplementing soft intradermal, subcutaneous or periosteal tissues of a patient, said method having the following steps:
inserting the extremity of the needle of the device of claim 1 into said tissues of said patient;
activation of the rotary motor of said device to displace said piston by means of the drive mechanism transforming the rotary motion of said rotary motor into a linear motion of said piston to expel through said needle a cosmetic and/or therapeutic product located in said container, wherein the variations in the rate of said cosmetic and/or therapeutic product through said needle are absorbed by said damping element.

14. A treatment method intended for rheumatology, phlebology, for the treatment of hyperhidrosis or for the obturation of the vein of a patient, including the injection of toxins, of an active agent or of a medicament to said patient, said method including the following steps:
insertion of the extremity of the needle of the device of claim 1 into a cartilage, an articulation, a vein, an intra-ocular cavity or an extra-ocular surgical cavity of said patient;
activation of the rotary motor of said device for moving said piston by means of a drive mechanism, which converts the rotary motion of the rotary motor into a linear motion of said piston to expel through said needle toxins, an active agent or of a medicament located in said container, wherein the variations in the rate of said toxins, active agent or medicament through said needle are absorbed by said damping element.

15. A method for filling soft intra-dermal, subcutaneous or periosteal tissues of a patient including the injection into said tissues of said patient of a filler product by means of the device of claim 1.

16. The method of claim 15, wherein said injection is a continuous injection.

17. The method of claim 15, wherein said injection is a serial puncture injection.

18. A method for the rejuvenation of soft intra-dermal, subcutaneous or periosteal tissues of a patient including the injection into said tissues of said patient of a rejuvenation product by means of the device of claim 1.

19. The method of claim 18, wherein said injection is a continuous injection.

20. The method of claim 18, wherein said injection is a serial puncture injection.

21. A therapeutic method including the injection of toxins, of an active agent or of a medicament into a cartilage, an articulation, a vein, an intra-ocular cavity or an extra-ocular surgical cavity of a patient, said injection step being performed by means of the device of claim 1.

22. The method of claim 21, wherein said injection is a continuous injection.

23. The method of claim 21, wherein said injection is a serial puncture injection.
